(19) United States
(12) Patent Application Publication
(10) Pub. No.: US 2016/0263208 A1
(43) Pub. Date: Sep. 15, 2016

(54) INFLUENZA VACCINE FOR SKIN IMMUNIZATION

(71) Applicant: Sporos Therapeutics, LLC, Bothell, WA (US)

(72) Inventors: Ioanna Skountzou, Atlanta, GA (US); E. Stein Esser, Atlanta, GA (US); Richard Compans, Atlanta, GA (US); Shaguna Seth, Bothell, WA (US)

(21) Appl. No.: 15/066,979

(22) Filed: Mar. 10, 2016

Related U.S. Application Data
(60) Provisional application No. 62/130,748, filed on Mar. 10, 2015.

(51) Int. Cl. A61K 39/145; C12N 7/00; A61K 9/00

(52) U.S. Cl. CPC A61K 39/145; A61K 9/0021; C12N 7/00; A61K 2039/54

(57) ABSTRACT

A method for preventing, treating or reducing the effects of influenza infection in a subject by administering an influenza vaccine to the skin of the subject. The administration can be intradermal or by microneedle patch. The method can provide increased protection for pregnant subjects and their offspring, and have an increased duration of action over conventional intramuscular vaccination.

### BACKGROUND OF THE INVENTION

Influenza infection can be life-threatening for pregnant women and their newborns, and hence of great public health concern.

Pregnancy is a risk factor for severe complications from influenza virus infection. Influenza infections during the second and third trimester of pregnancy showed a five-fold increase in cardiopulmonary complications, morbidity and mortality compared to a non-pregnant population. As for influenza infection-related complications in fetuses and neonates include increased risk of miscarriage, stillbirth, neonatal death, preterm birth, and low birth weight. Prematurity is largest single cause in death in children under five. Notably it causes about 17% of all deaths in children under five, and about 35% percent of neonatal deaths, which translates to hundreds of thousands lives every year.

Unvaccinated pregnant women should receive influenza vaccine, preferably in the third or late second (after 20 weeks gestation) trimester in order to optimize the concentration of maternal antibodies transferred to the fetus. However, only half of pregnant women receive influenza vaccines. While it is not recommended to administer influenza vaccine to infants younger than six months of age, influenza vaccine given to pregnant women can be effective in preventing hospitalization of infants six months or younger due to influenza-related disease.

Vaccination of pregnant women has a “two-in-one” benefit. Mothers can transfer protective antibodies through placenta or through breast milk providing protection in infants until they can be immunized. Immunization not only confers protective immunity to the mother, but also provides a passive immune response to the infant, prior to the development of its own antibodies. Maternal antibodies are transferred through the umbilical cord blood during fetal development and breast milk during infant nursing.

Although immunization of pregnant women is one of the most effective means of preventing maternal and infant mortality and morbidity, the availability of vaccines in low-resource settings is limited. Lack of cold storage chain availability is a key limitation.

Given the needs of pregnant women and their fetuses for ante-natal care, especially in developing countries, there is a need for an easy-to-administer, thermostable, vaccine-containing patch that generates no sharps waste.

What is needed are methods, compositions and devices for influenza immunization.

### BRIEF SUMMARY

This invention relates to the field of vaccines for influenza.

Embodiments of this invention include:

A method for preventing, treating or reducing the effects of influenza infection in a subject, comprising administering an influenza vaccine to the skin of the subject.

The method above, wherein the influenza vaccine does not require cold storage or cold transport.

The method above, wherein the influenza vaccine is an influenza subunit vaccine.

The method above, wherein the influenza vaccine comprises an influenza subunit vaccine at an effective dose of from 1.5 to 5 μg of HA.

The method above, wherein the influenza vaccine comprises an influenza subunit vaccine at an effective dose of 2.5 μg of HA.

The method above, wherein the administration is intradermal or microneedle.

The method above, wherein the microneedle administration is a microneedle patch.

The method above, wherein the subject is pregnant.

The method above, wherein offspring born to immunized mothers have higher levels of specific anti-influenza antibodies in sera than offspring born to mothers immunized with a double dose of the same vaccine via an intramuscular route.

The method above, further comprising:

administering an influenza vaccine by placing a microneedle patch containing the vaccine on the skin of the subject;

holding the patch in place to allow the vaccine to dissolve into the skin.

The method above, wherein the microneedles of the patch penetrate the skin and the patch is held in place for at least 10 minutes.

The method above, wherein no vaccine reconstitution is required prior to administration, and the microneedle vaccination patch is stored at ambient temperature.

The method above, wherein at least a three-fold higher level of influenza-specific antibodies is induced in pregnant subjects than for intramuscular administration in pregnant subjects.

The method above, wherein at least a five-fold higher level of influenza-specific antibodies is induced in non-pregnant subjects than for intramuscular administration in non-pregnant subjects.

The method above, wherein at least a six-fold higher level of influenza-specific antibodies IgG2a is induced in a pregnant subject as compared to intramuscular administration in a non-pregnant subject.

The method above, wherein the duration of protection against influenza infection is greater than for intramuscular administration.

The method above, wherein the duration of protection against influenza infection is twice as long as for intramuscular administration.

### BRIEF DESCRIPTION OF THE DRAWINGS

FIG. 1A: Pregnant mouse model and experimental design. Representative pictures showing mouse vaginal opening during (top left) non-estrous stage, (top right) estrous stage, and with a plug (lower).

FIG. 1B: Pregnant mouse model and experimental design. Following mating, body weight of each mouse was monitored to ensure the mice were pregnant. Each pregnant mouse was then immunized on day 11-13 after mating/presence of a plug. Typically each mouse delivered pups between 20-22 day of gestation.

FIG. 1C: Pregnant mouse model and experimental design. Experimental design of the study. Pregnant BALB/c mice were immunized (11-13 day of gestation) with A/Brisbane/59/07 H1N1 vaccine via intramuscular route or cutaneously using either fish gelatin microneedles encapsulating the vaccine or hypodermic needles delivering vaccine in solution. Non-pregnant female mice were also immunized with the same vaccine via both routes. On day 30 after immunization mice were bleed and 2 days later were challenged with homologous virus. The pups were weaned three weeks after birth and bleed on days 21 (week 3), 28 (week 4), 35 (week 5), 42 (week 6), 56 (week 8), 70 (week 10) and 84 (week 12). A group of 6 week old pups was challenged with A/Brisbane/59/07 H1N1 virus 6 weeks after birth.

FIG. 1D: Pregnant mouse model and experimental design. Overall data after 7 matings (31% mice did not getting pregnant after being in a cage with a male 7 times for 3-4 days each time).

FIG. 2A: Humoral immune responses in pregnant mice immunized with A/Brisbane/59/07 H1N1 subunit vaccine encapsulated in dissolving microneedles (MN) or intramuscularly (IM). Anti-influenza binding antibodies were determined by ELISA in sera collected from mice 28 days after immunization. FIG. 2A shows IgG antibody titers.

FIG. 2B: Humoral immune responses in pregnant mice immunized with A/Brisbane/59/07 H1N1 subunit vaccine encapsulated in dissolving microneedles (MN) or intramuscularly (IM). Anti-influenza binding antibodies were determined by ELISA in sera collected from mice 28 days after immunization. FIG. 2B shows IgG1 antibody titers.

FIG. 2C: Humoral immune responses in pregnant mice immunized with A/Brisbane/59/07 H1N1 subunit vaccine encapsulated in dissolving microneedles (MN) or intramuscularly (IM). Anti-influenza binding antibodies were determined by ELISA in sera collected from mice 28 days after immunization. FIG. 2C shows IgG2a antibody titers.

FIG. 2D: Humoral immune responses in pregnant mice immunized with A/Brisbane/59/07 H1N1 subunit vaccine encapsulated in dissolving microneedles (MN) or intramuscularly (IM). Anti-influenza binding antibodies were determined by ELISA in sera collected from mice 28 days after immunization. FIG. 2D shows Hemagglutination inhibition (HAI) in sera collected 28 days after immunization. Values are expressed as geometric mean with a ±95% confidence interval (n=8-20). [IM, n=8; MN, n=14 (pregnant mice), n=20 (non-pregnant mice)].

FIG. 2E: Humoral immune responses in pregnant mice immunized with A/Brisbane/59/07 H1N1 subunit vaccine encapsulated in dissolving microneedles (MN) or intramuscularly (IM). Anti-influenza binding antibodies were determined by ELISA in sera collected from mice 28 days after immunization. FIG. 2E shows neutralizing antibody (NT) titers in sera collected 28 days after immunization. Values are expressed as geometric mean with a ±95% confidence interval (n=8-20). [IM, n=8; MN, n=14 (pregnant mice), n=20 (non-pregnant mice)].

FIG. 2F: Summary of fold changes and statistical differences in humoral responses in pregnant on non-pregnant mice immunized via intramuscular or cutaneous routes.

FIG. 3A: Protective immunity after lethal challenge with homologous of mice immunized during pregnancy. Immunized groups were challenged with mouse adapted A/Brisbane/59/07 (H1N1) virus 4 weeks after immunization. FIG. 3A shows Body weight changes after challenge with 5×LD₅₀ of virus were monitored for 14 days (5-14 mice/group).

FIG. 3B: Protective immunity after lethal challenge with homologous of mice immunized during pregnancy. Immunized groups were challenged with mouse adapted A/Brisbane/59/07 (H1N1) virus 4 weeks after immunization. A separate cohort of immunized mice was challenged with the same infectious dose 80 days after immunization. FIG. 3B shows Body weight changes.

FIG. 3C: Protective immunity after lethal challenge with homologous of mice immunized during pregnancy. Immunized groups were challenged with mouse adapted A/Brisbane/59/07 (H1N1) virus 4 weeks after immunization. FIG. 3C shows survival rates after challenge with 5×LD₅₀ of virus were monitored for 14 days (5-14 mice/group).

FIG. 3D: Protective immunity after lethal challenge with homologous of mice immunized during pregnancy. Immunized groups were challenged with mouse adapted A/Brisbane/59/07 (H1N1) virus 4 weeks after immunization. A separate cohort of immunized mice was challenged with the same infectious dose 80 days after immunization. FIG. 3D shows survival rates.

FIG. 4A: Humoral immune responses in pups born to mothers immunized with A/Brisbane/59/07 H1N1 subunit vaccine via intramuscular and transcutaneous routes during pregnancy. Anti-influenza binding antibodies were determined by ELISA in sera collected from pups on week 3, 4, 5, 6 after birth. FIG. 4A shows IgG antibody titers.

FIG. 4B: Humoral immune responses in pups born to mothers immunized with A/Brisbane/59/07 H1N1 subunit vaccine via intramuscular and transcutaneous routes during pregnancy. Anti-influenza binding antibodies were determined by ELISA in sera collected from pups on week 3, 4, 5, 6 after birth. FIG. 4B shows IgG1 antibody titers.

FIG. 4C: Humoral immune responses in pups born to mothers immunized with A/Brisbane/59/07 H1N1 subunit vaccine via intramuscular and transcutaneous routes during pregnancy. Anti-influenza binding antibodies were determined by ELISA in sera collected from pups on week 3, 4, 5, 6 after birth. FIG. 4C shows IgG2c antibody titers.

FIG. 4D: Humoral immune responses in pups born to mothers immunized with A/Brisbane/59/07 H1N1 subunit vaccine via intramuscular and transcutaneous routes during pregnancy. Anti-influenza binding antibodies were determined by ELISA in sera collected from pups on week 3, 4, 5, 6 after birth. FIG. 4D shows Hemagglutination inhibition (HAI) in sera collected 28 days after immunization.

FIG. 4E: Humoral immune responses in pups born to mothers immunized with A/Brisbane/59/07 H1N1 subunit vaccine via intramuscular and transcutaneous routes during pregnancy. Anti-influenza binding antibodies were determined by ELISA in sera collected from pups on week 3, 4, 5, 6 after birth. FIG. 4E shows neutralizing antibody (NT) titers in sera collected 28 days after immunization.

FIG. 5A: Protective immunity after challenge with A/Brisbane(H1N1) virus of pups born to mothers immunized during pregnancy. Pups were challenged with 5×LD50 of mouse adapted A/Brisbane(H1N1) virus 6 weeks after birth. FIG. 5A shows survival rates monitored for 14 days (5 mice/group).

FIG. 5B: Protective immunity after challenge with A/Brisbane(H1N1) virus of pups born to mothers immunized during pregnancy. Pups were challenged with 5×LD50 (A and B) or 5×LD50 (c and d) of mouse adapted A/Brisbane(H1N1) virus 6 weeks after birth. FIG. 5B shows Body weight changes monitored for 14 days (5 mice/group).

FIG. 6A: Evaluation of humoral responses and neutralizing antibody titers of offspring birthed to mice immunized during pregnancy. Serum samples from offspring of mothers vaccinated during pregnancy either intradermally (ID) or intramuscularly (IM) were collected when the pups were 3, 6, and 8 weeks of age. FIG. 6A shows the samples were analyzed against A/Brisbane/59/2007 for the levels of total serum IgG antibody titers by ELISA.

FIG. 6B: Evaluation of humoral responses and neutralizing antibody titers of offspring birthed to mice immunized during pregnancy. Serum samples from offspring of mothers vaccinated during pregnancy either intradermally (ID) or intramuscularly (IM) were collected when the pups were 3, 6, and 8 weeks of age. FIG. 6B shows the samples were analyzed against A/Brisbane/59/2007 for the levels of IgG isotypes, IgG1.

FIG. 6C: Evaluation of humoral responses and neutralizing antibody titers of offspring birthed to mice immunized during pregnancy. Serum samples from offspring of mothers vaccinated during pregnancy either intradermally (ID) or intramuscularly (IM) were collected when the pups were 3, 6, and 8 weeks of age. FIG. 6C shows the samples were analyzed against A/Brisbane/59/2007 for the levels of IgG2a by ELISA.

FIG. 6D: Evaluation of humoral responses and neutralizing antibody titers of offspring birthed to mice immunized during pregnancy. Serum samples from offspring of mothers vaccinated during pregnancy either intradermally (ID) or intramuscularly (IM) were collected when the pups were 3, 6, and 8 weeks of age. FIG. 6D shows the samples were analyzed against A/Brisbane/59/2007 for the levels of neutralizing antibody titers by microneutralization assay.

FIG. 7A: Protective immune response in offspring after lethal challenge with A/Brisbane/59/07 H1N1 virus. Six week old mice born from mothers immunized during pregnancy by ID or IM delivery were challenged with 3×LD₅₀ A/Brisbane/59/07 H1N1 virus. FIG. 7A shows Survival rates. Data points represent the mean±SEM. Statistics were done using a two-way ANOVA with Bonferroni post-tests. *p<0.05; ***p<0.001.

FIG. 7B: Protective immune response in offspring after lethal challenge with A/Brisbane/59/07 H1N1 virus. Six week old mice born from mothers immunized during pregnancy by ID or IM delivery were challenged with 3×LD₅₀ A/Brisbane/59/07 H1N1 virus. FIG. 7B shows body weight changes. Data points represent the mean±SEM. Statistics were done using a two-way ANOVA with Bonferroni post-tests. *p<0.05; ***p<0.001.

### DETAILED DESCRIPTION OF THE INVENTION

This invention provides compositions, devices and methods for skin immunization for influenza. Skin immunization during pregnancy can be an alternative and efficient route to expand geographical vaccination coverage for protective immunity.

In some embodiments, skin vaccination during pregnancy via intradermal injection of inactivated influenza vaccine is more immunogenic than conventional intramuscular vaccine delivery.

Embodiments of this invention can provide a skin immunization having greater passive immune response amongst offspring, as well as significant protective efficacy against lethal infections, as compared to the offspring of those vaccinated by a conventional intramuscular route during pregnancy.

In some embodiments, the antibodies due to an immunization of this invention can last longer in serum than for conventional intramuscular vaccine delivery.

Without wishing to be bound by any particular theory, vaccine delivery in the skin may utilize antigen presenting cells (APCs) residing in the epidermis and the dermis. Skin vaccine delivery to APCs may provide an increased immune response compared to conventional intramuscular delivery.

Embodiments of this invention can provide an easy-to-administer influenza vaccination device, and methods for providing protective immunity superior to conventional immunization.

In some aspects, this disclosure provides microneedle influenza vaccination patches. The microneedle vaccination patches and methods of this invention can be dose sparing, and provide robust induction of immune responses at lower doses of vaccine.

In certain aspects, the microneedle vaccination patches and methods of this invention can advantageously be used for pregnant women.

In some embodiments, the microneedle vaccination patches can be dissolvable, and applied to skin with an adhesive backing. Thus, the microneedle vaccination patches can be easy to administer by a minimally trained personnel.

In further embodiments, no vaccine reconstitution may be required prior to administration, and the microneedle vaccination patches can be stored at room temperature.

In certain embodiments, vaccination of pregnant subjects with subunit H1N1 influenza vaccine using a microneedle vaccination patch of this invention can provide increased humoral immunity and protective immune responses, as compared to conventional immunization, i.e. intramuscular, even at a lower vaccine dose.

In further embodiments, microneedle vaccination patches and methods of this invention can be more effective in protecting offspring. Methods and devices of this invention can provide significantly higher influenza specific antibody titers and higher survival rates for offspring, as compared to offspring born to pregnant subjects immunized by a conventional intramuscular route with a double dose of the vaccine.

The microneedle vaccination patches of this invention can advantageously be made of dissolvable polymer that encapsulates the vaccine, and be self-applied to skin like a skin plaster.

A microneedle vaccination patch of this invention advantageously requires no vaccine reconstitution prior to administration.

Microneedles can be formulated to dissolve and release the vaccine within minutes once inserted in skin, thus simplifying waste management. The elimination of syringes and needles renders vaccine delivery safe, removing the risk of accidental infection by blood-borne pathogens.

A microneedle vaccine patch of this invention can be stored at room temperature, which advantageously eliminates the need for cold storage or cold transport.

Embodiments of this invention can provide microneedle vaccines for use in pregnant subjects.

A microneedle vaccine of this invention can provide increased efficacy in pregnant subjects over a conventional intramuscular vaccination route.

In certain aspects, delivery of an influenza microneedle vaccine of this disclosure can induce increased humoral immunity and protective immune responses as compared to conventional intramuscular immunization.

The influenza microneedle vaccine of this invention can be used without adverse effects in pregnant subjects. For example, an influenza microneedle vaccine of this invention can be used without producing physical marks on the skin at the site of immunization, without behavioral changes, without body weight decrease, and without premature offspring delivery.

In addition to the induction of robust immune responses, significant dose sparing can be provided when using an influenza microneedle vaccine of this invention. Pregnant subjects vaccinated with an influenza microneedle vaccine of this invention and their offspring may have higher anti-influenza antibody titers attributing to superior protective immunity as compared to subjects receiving a conventional vaccine via the intramuscular route.

In general, lower immune responses following influenza vaccination can occur for subjects vaccinated during pregnancy compared to non-pregnant subjects, regardless of the route of administration. This may be due to multiple changes in a pregnant woman's immune system during pregnancy to adapt and tolerate a genetically different fetus.

In some embodiments, a microneedle vaccine of this invention can provide surprisingly increased protection for pregnant subjects over a conventional intramuscular vaccine.

In further embodiments, a microneedle influenza vaccine of this invention can provide protection to offspring. Offspring born to mothers immunized using a microneedle influenza vaccine of this invention can have higher levels of specific anti-influenza antibodies in sera than offspring born to mothers immunized with a double dose of the same vaccine via the intramuscular route.

A subject of this invention can be a human or animal.

### EXAMPLE 1

Specific antibody titers, including HAI and neutralizing antibody titers, which are considered as the correlates of protective immunity, were measured and compared in the sera from microneedle and intramuscularly vaccinated both pregnant and non-pregnant mice.

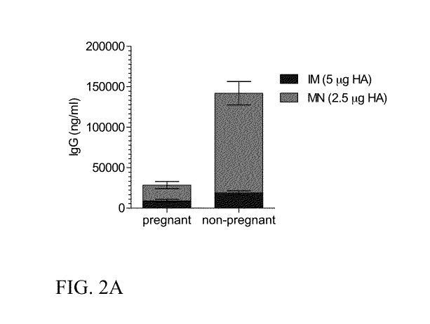

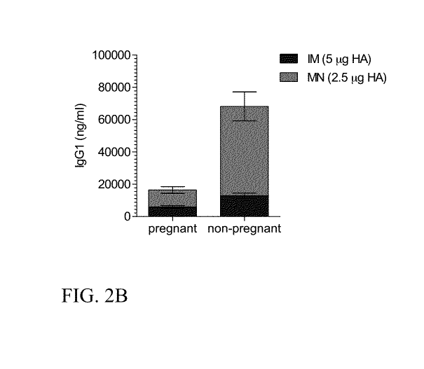

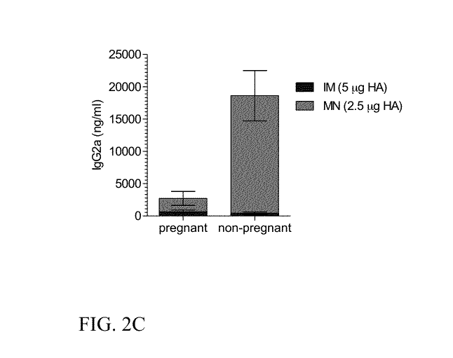

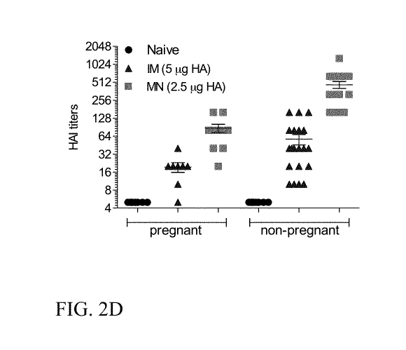

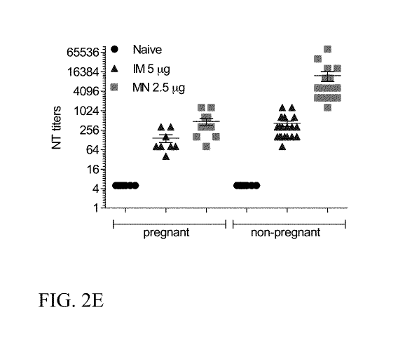

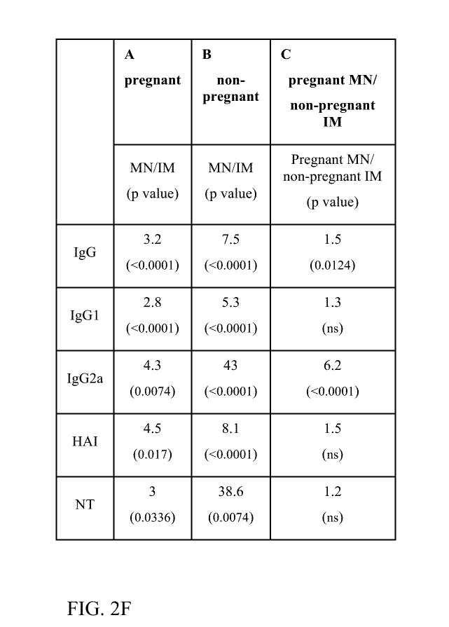

As shown in FIG. 2F, the immune responses to microneedle vaccination were superior to those observed in mice that were immunized by the conventional intramuscular route with a double dose of the vaccine, whether the mice were pregnant or non-pregnant (columns A and B).

In addition, as shown in FIG. 2F, antibody production in pregnant mice vaccinated with microneedles was superior to antibody production in non-pregnant mice vaccinated intramuscularly (column C).

Further, as shown in FIG. 2F, microneedle vaccination resulted in a significant increase of IgG2a antibodies in both pregnant and non-pregnant mice. This may reflect an increase in Thl immune responses.

### EXAMPLE 2

In this study, it is shown that pups born to mothers immunized using a microneedle patch encapsulating subunit influenza vaccine had higher levels of specific anti-influenza antibodies in sera than mice that were born to mothers immunized with a double dose of the same vaccine via the intramuscular route.

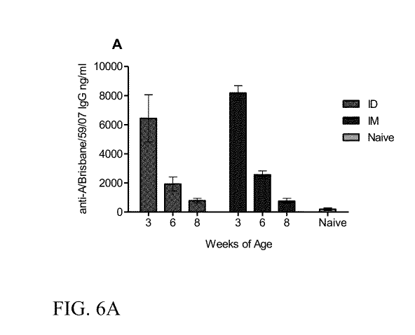

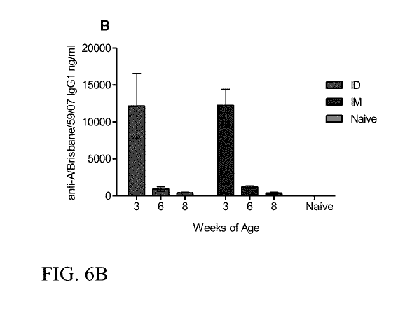

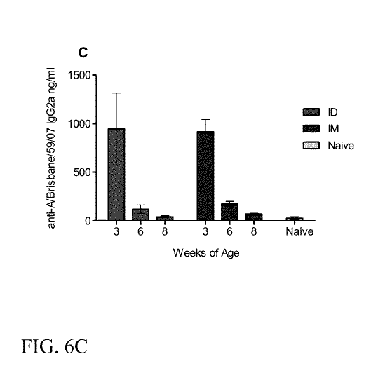

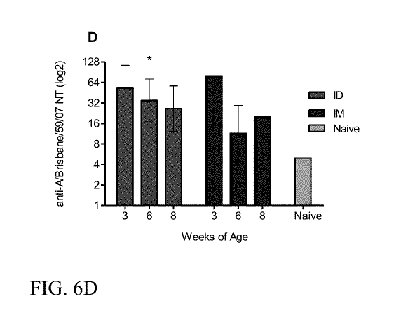

In this experiment, female adult BALB/c mice in estrus were paired with male mice in harem housing conditions. Females were observed daily for the presence of a copulation plug to indicate mating had taken place. In order to confirm pregnancies, body weights of the females were recorded daily. At approximately day 12 of gestation, pregnant females gained 20 to 25% of their original body weight and were immunized with 2.5 ug HA of subunit A/Brisbane/59/07 H1N1 vaccine via intradermal (ID) or intramuscular (IM) injection. Pregnant females gave birth to offspring about one week later between the nineteenth and twenty second day of total gestation. At three weeks old, offspring were weaned from their mothers and serum was collected from the pups at 3, 6, and 8 weeks of age by means of submandibular bleeding in order to analyze the passive immune response. Virus-specific IgG antibody levels were determined by ELISA. For total IgG (FIG. 6A) and its isotypes, IgG1 (FIG. 6B) and IgG2a (FIG. 6C), a noticeable reduction in antibody titers was observed as time progressed after weaning.

Neutralizing antibody titers can provide a useful measure of protective immunity to influenza virus. Neutralizing antibody titers were determined in heat inactivated sera by microneutralization assay using 100 TCID₅₀/well of A/Brisbane/59/07 virus. According to the CDC, a neutralization titer of 1:40 is associated with at least a 50% reduction in risk for influenza infection in the pediatric population. At the time of weaning (week 3), neutralizing antibody titers for both ID and IM offspring were above the protective titer 1:40 (FIG. 6D). At six weeks of age, neutralizing titers for both groups declined, however the neutralizing antibody titer for the ID offspring (1:35) was three-fold greater than the IM offspring (p<0.05).

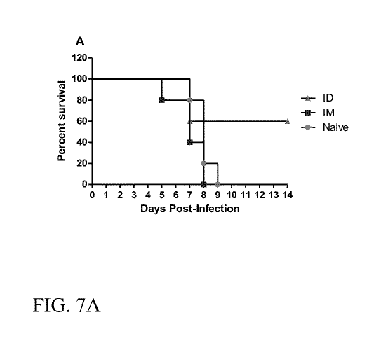

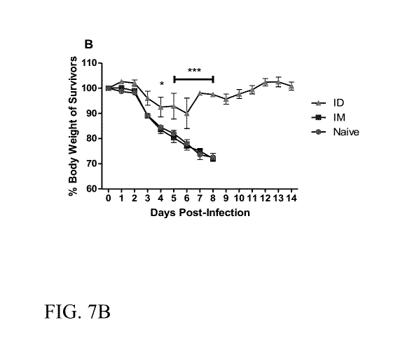

In order to determine if the offspring of mice vaccinated during pregnancy were protected from a lethal challenge with homologous virus, six-week old mice (21 days post-weaning) were infected intranasally with 3×LD₅₀ of mouse adapted A/Brisbane59/07 H1N1 virus while under isoflurane anesthetic. The mice were monitored for two weeks for signs of morbidity and mortality. Signs observed include: body weight changes, dehydration, lethargy, hunched posture, and mortality. Weight loss exceeding 25% was used as the experimental end point, at which mice were euthanized according to IACUC guidelines. The survival rate for pups born from ID vaccinated mothers was 60%, while the survival rate for pups born from IM vaccinated and naive mothers was 0% (FIG. 7A). Furthermore, the ID offspring lost a significantly less amount of body weight during the observation period post-infection when compared to the IM offspring. The ID young lost at most an average of 10% their pre-challenge body weight while both the IM and naive offspring cohorts lost more than 25% of their body mass hence they were euthanized (p<0.001) (FIG. 7B).

These results show that pups born to mothers immunized using a microneedle influenza vaccine had higher levels of specific anti-influenza antibodies in sera than mice that were born to mothers immunized with a double dose of the same vaccine via the intramuscular route.

The highest antibody titers were observed in 3 week-old pups that were still housed with the mothers, and gradual decrease was observed with time following separation from mothers.

Pups born to microneedle immunized mothers maintained significantly higher antibody titers at any tested time and, for up to nine weeks after weaning, than pups born to intramuscularly immunized mice. The data here show the long-lasting passive immunity in offspring. Consistently, higher survival rates were observed in weaned pups born to microneedle immunized mice.

In sum, skin immunization during pregnancy is a novel and effective approach to boost the immune response in both mother and fetus. Skin immunizations against A/Brisbane/59/07 in pregnant mouse models were shown to significantly increase the amount of neutralizing antibodies in offspring after weaning and better protect the young from lethal challenges with H1N1 influenza than conventional (intramuscular) vaccination.

### EXAMPLE 3

Cells and virus stocks. Madin-Darby canine kidney (MDCK) cells (CCL 34, ATCC, Manassas, Va.) were maintained in Dulbecco's Modified Eagle's Medium (Mediatech, Herndon, Va.) containing 10% fetal bovine serum (Hyclone, Thermo Scientific, Rockford, Ill). Influenza virus stocks (A/Brisbane/59/07 (H1N1)) were propagated in MDCK cells. The hemagglutination (HA) activity was determined using turkey blood cells (LAMPIRE, Pipersville, Pa.). Mouse-adapted virus was obtained by serially passaging in lungs of BALB/c mice, and titers were determined by plaque assay. The LD₅₀ was determined using Reed-Munch formula.

### EXAMPLE 4

Animals. Eight week-old female BALB/c mice were purchased from Harlan Laboratories (Tampa, Fla.). All, mice were bred and housed in a biosafety level 1 facility for immunizations whereas infections took place in a biosafety level 2 at Emory University Whitehead animal facility. All mice used for this study was 10-14 weeks old.

### EXAMPLE 5

Fish gelatin microneedle preparation. The vaccine formulation consisting of concentrated monovalent vaccine, sucrose, fish gelatin and sulforhodamine B dye (Sigma Aldrich) in 100 mM dibasic potassium phosphate buffer pH 7.4 was cast onto a PDMS mold (100 microneedles per array; each microneedle measuring 700 μm in length and 200 μm in width at the base). Vacuum was applied to ensure that the formulation filled the entire microneedle cavity and the formulation was allowed to air dry. In the second step, the backing formulation consisting of fish gelatin and sucrose in 100 mM dibasic potassium phosphate buffer pH 7.4 was cast onto the mold under vacuum and subsequently dried at room temperature overnight before demolding the microneedle patch.

### EXAMPLE 6

H1N1 A/Brisbane/59/07 subunit vaccine was concentrated and buffer exchanged with 100 mM dibasic potassium phosphate using spin filters (Amicon, Billerica, Mass. and Vivaspin, Sartorius Stedium, Germany). Protein concentration was measured using bicinchoninic acid assay (BCA) with bovine serum albumin as the standard (Thermo, Mass., USA). Hemagglutinin content was measured by single radial immunodiffusion (SRID).

Immunizations, challenge and sample collection. Two groups of mice were immunized. One was 10 week-old non-pregnant female mice and the second was 10-14 week-old pregnant mice mice. Pregnancy was determined if the animals had a visible plug during mating and/or displayed significant body weight increase 11-13 days after mating. Immunizations of pregnant mice were done between days 11-13 after mating. Prior to immunization, each mouse was shaved using clippers and Nair. Dissolving microneedle patches encapsulating the vaccine were inserted into the skin and left in place for 10 min. Since vaccine delivery efficiency is 60%, the vaccine dose encapsulated in the patch was adjusted to deliver the desired HA concentration (2.5 μg HA). Another group of pregnant mice was immunized with 5 μg HA intramuscularly (IM). Animals were bled (cheek bleed) 28 days post-immunization. The pups were weaned 3 weeks after birth (day 21) and bleed (cheek bleed) on days 21, 28, 35, 42, 56, 70, 84 after birth. For challenge, adult mice and pups were infected intranasally with 5×LD₅₀of mouse adapted A/Brisbane/59/07 virus under isoflurane anesthesia. The animals were monitored for 14 days for body weight changes, fever, hunched posture, and mortality. Weight loss exceeding 25% was used as the experimental end point, at which mice were euthanized according to IACUC guidelines. All studies were approved by Emory University's Institutional Animal Care and Use Committee.

Humoral immune responses. Virus-specific antibody levels were determined by ELISA. Hemagglutination inhibition titers (HAI) were assessed using the WHO protocol. Neutralizing antibody titers were determined in heat inactivated sera by microneutralization assay using 100 TCID₅₀/well of A/Brisbane/59/07 virus.

Statistics. The statistical significance of differences was calculated by two-tailed unpaired Student's t-test and one-way ANOVA including Bonferronis's multiple comparison test. A p value less than 0.05 was considered significant.

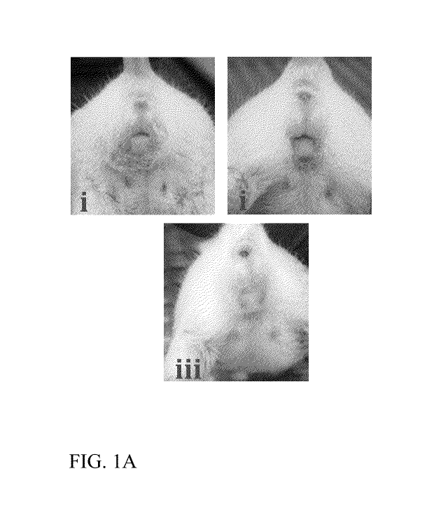

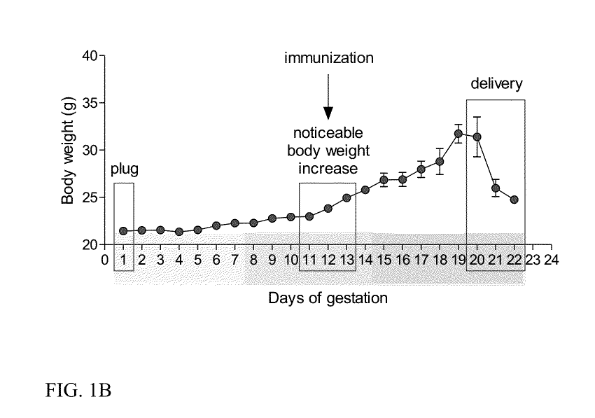

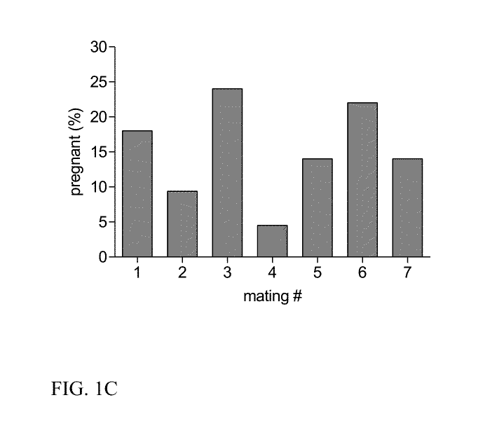

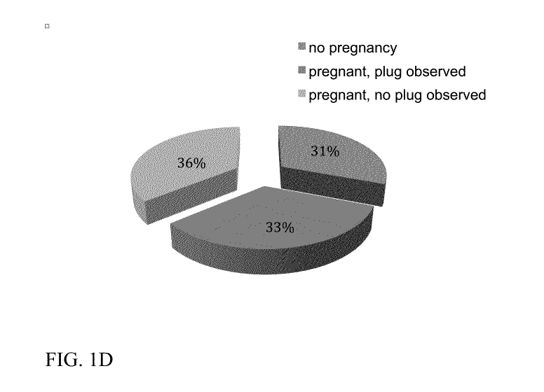

Pregnant mouse model and experimental design. Mouse estrous cycle can be divided into four stages, estrous, metesestrous, diestrous, and proestrous, which can be determined by visual examination of vaginal opening, and by vaginal cytology. Estrous cycle in a laboratory a mouse is 4-6 days, but the estrous phase lasts for only 6-8 h. Different mouse strains have different reproductive fitness and the BALB/c mice are one of the least efficient breeders. Breading cages were set up with two females and a male for 3 days and the presence of a plug was observed. Mating was repeated in 4-5 day intervals to accurately time each pregnancy. Each female was monitored daily for body weight change to confirm pregnancy. The chance of BALB/c mouse pair to produce offspring is about 50%. In this study a breeding protocol was established allowing precise estimation of stage of pregnancy and gestational age of fetuses. Tagged female mice were used for breeding. Three females were housed with one male for 3-4 days and during that time each female was daily observed for vaginal opening, indication of estrous phase, and the presence of a copulation plug (FIG. 1A). Once the male mated with female the excess of sperm forms a copulation plug. The plug is visible after visual examination of vulva and will persist 16-24 h after copulation. As an additional means of determining pregnancy, each female was daily monitored for body weight change (FIG. 1B). Pregnant mice were placed in separate cages, two females per cage, and the remaining mice were mated again. Sequential matings were performed, each time the females were housed with a male for 3-4 days in 4-5 day intervals. We observed that the BALB/c mice breed very poorly, with breading efficiency of less than 25% after a single mating (FIG. 1C). Overall, after 7 matings 69% of mice become pregnant. Plug was observed in about half of all the pregnant mice.

FIG. 1D shows Pregnant mouse model and experimental design. Overall data after 7 matings (31% mice did not getting pregnant after being in a cage with a male 7 times for 3-4 days each time).

Pregnant females had noticeable body weight increase between days 11-13 after mating, hence that time point was used for immunizations. Pregnant (11-13 day of gestation) or non-pregnant mice were vaccinated via either the intramuscular route or using dissolving microneedles encapsulating A/Brisbane/59/07 (H1N1) subunit vaccine. Intramuscularly vaccinated mice received total of 5 μg of HA. Since we have previously observed significant dose sparing when using microneedles, mice immunized via microneedles received a reduced dose of the same vaccine, on average 2.5 μg of HA. Following immunization body weights were continued to be recorded until delivery on 20-22 day after mating. We did not observe any adverse effects on the pregnancy following either intramuscular or microneedle immunization. Microneedle insertion did not leave a mark at the site of vaccination, none of the animals had a premature delivery (data not shown), and no fluctuations in the body weight were observed after vaccination. Twenty eight days after immunization, which corresponded to about 21 days after delivery, the mothers were bled and then challenged with homologous virus. The pups were weaned three weeks after birth and bleed on days 21 (week 3), 28 (week 4), 35 (week 5), 42 (week 6), 56 (week 8), 70 (week 10) and 84 (week 12). A group of 6 week old pups was challenged with mouse A/Brisbane H1N1 virus.

Pregnant and non-pregnant mice immunized with half dose of the vaccine via microneedles have higher humoral responses than mice immunized intramuscularly. Serum collected 28 days after immunization was analyzed by ELISA to determine levels of vaccine specific antibody titers. IgG, IgG1, and IgG2a antibody titers were about 3-4 folds higher in pregnant mice immunized with 2.5 μg of HA via dissolvable microneedles as compared to pregnant mice immunized with 5 μg of HA via the standard intramuscular route (p<0.0001) (FIGS. 2A, 2B, 2C, and FIG. 2F). Non-pregnant mice immunized with microneedles had 7.5 and 5.3, times higher IgG, and IgG1 antibody titers, respectively, as compared to non-pregnant mice immunized intramuscularly with a double dose of the vaccine (p<0.0001). Interestingly, non-pregnant mice immunized with microneedles had 43 fold higher IgG2a antibody titers as compared with intramuscularly immunized non-pregnant mice indicating that microneedle immunization promotes Thl responses in non-pregnant. HAI and neutralizing antibody titers were also determined since they are considered to be indicative of protective immunity. Higher HAI and NT titers were observed in mice immunized with microneedles. About 4.5 (pregnant, p<0.0001) and 3 (non-pregnant, p<0.00336) fold change was observed in HAI titers in mice immunized with microneedles as compared to mice immunized intramuscularly, and 8.1 (pregnant, p<0.0001) and 38.6 (non-pregnant, p<0.0074) fold higher neutralizing antibody titers were observed in mice immunized with microneedles as compared to mice immunized intramuscularly. It is important to note that significant dose sparing was observed when microneedles were used for vaccine delivery. Although, only about half dose of the vaccine was delivered to the skin of both pregnant and non-pregnant mice, higher humoral immune responses were observed than in mice immunized with full dose of the vaccine via the intramuscular route.

During pregnancy adaptations are made to the immune system to tolerate a genetically different fetus, many of which have a suppressive effect on mother's immune system. Pregnant mice, immunized via either the intramuscular or transcutaneous routes, had significantly lower humoral immune responses as compared to non-pregnant mice (FIG. 2F). At least four fold lower influenza specific antibody titers were observed in pregnant mice immunized with microneedles as compared to non-pregnant mice immunized via the same immunization route. The difference was most pronounced when comparing the neutralizing titers in those two groups. The non-pregnant mice immunized with microneedles had 21.6 fold higher neutralizing antibody titers than the pregnant mice, while 2.6 fold higher neutralizing antibody titers were observed in non-pregnant mice immunized intramuscularly as compared to the pregnant group. Although the results of this study show that immune response to the vaccine is suppressed in pregnant mice, half dose of the vaccine delivered via microneedles in pregnant mice still induced better response to the vaccine than the response to intramuscular vaccination in both pregnant and non-pregnant mice (fold changes are summarized in FIG. 2F). When antibody titers in pregnant and non-pregnant mice immunized via the intramuscular route were compared also higher titers were observed in non-pregnant mice; at least two fold higher IgG, IgG1, HAI and NT titer were detected. In contrast to what was observed between the pregnant and non-pregnant mice vaccinated via microneedles, IgG2a titers in non-pregnant mice immunized intramuscularly were not higher than those in pregnant mice. Overall, these results indicate that microneedle immunization in non-pregnant mice is more efficient in the induction of specific IgG2a and neutralizing antibody production than intramuscular vaccination.

FIG. 2D shows Humoral immune responses in pregnant mice immunized with A/Brisbane/59/07 H1N1 subunit vaccine encapsulated in dissolving microneedles (MN) or intramuscularly (IM). Anti-influenza binding antibodies were determined by ELISA in sera collected from mice 28 days after immunization. FIG. 2D shows Hemagglutination inhibition (HAI) in sera collected 28 days after immunization. Values are expressed as geometric mean with a ±95% confidence interval (n =8-20). [IM, n=8; MN, n=14 (pregnant mice), n=20 (non-pregnant mice)].

FIG. 2E shows Humoral immune responses in pregnant mice immunized with A/Brisbane/59/07 H1N1 subunit vaccine encapsulated in dissolving microneedles (MN) or intramuscularly (IM). Anti-influenza binding antibodies were determined by ELISA in sera collected from mice 28 days after immunization. FIG. 2E shows neutralizing antibody (NT) titers in sera collected 28 days after immunization. Values are expressed as geometric mean with a ±95% confidence interval (n =8-20). [IM, n=8; MN, n=14 (pregnant mice), n=20 (non-pregnant mice)].

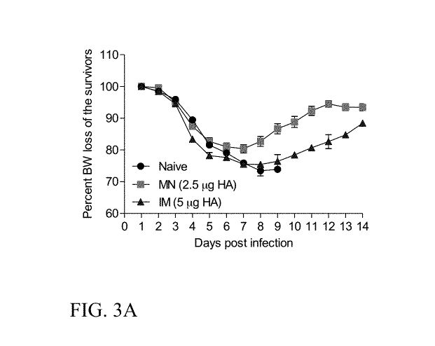

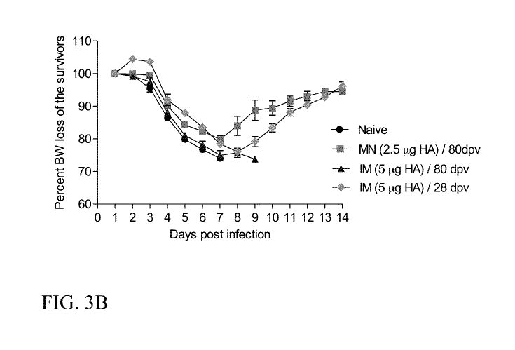

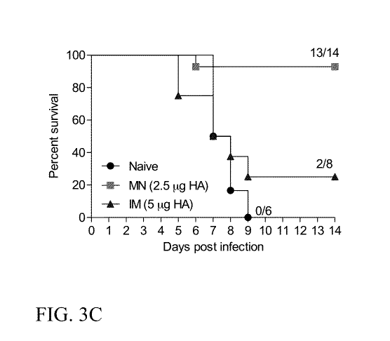

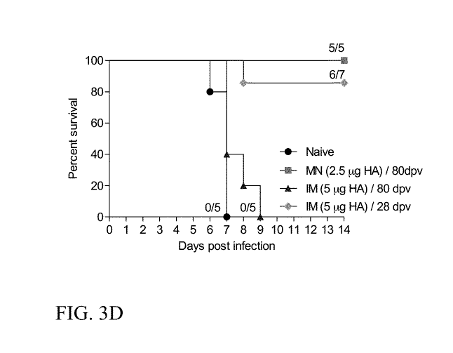

Pregnant and non-pregnant mice immunized with half dose of the vaccine via microneedles have higher protective immunity than mice immunized intramuscularly. To determine if mice vaccinated during pregnancy are protected from lethal challenge with homologous virus, the animals were infected with 5×LD50 of A/Brisbane/59/07 (H1N1) virus and monitored for morbidity and mortality for 14 days. All infected animals displayed signs of disease including ruffled hair, hunched posture, and body weight loss (FIGS. 3A and 3B). Although mice immunized with microneedles during pregnancy lost up to 20% body weight by day 7 after infection 13 out of 14 infected animals survived the challenge (FIGS. 3C and 3D). In contrast to the 93% survival rate for the microneedle immunized group of mice, 25% survival rate was observed in mice that were immunized intramuscularly. Based on high HAI and neutralizing titers observed 4 weeks after vaccination of non-pregnant mice we decided to challenge the mice at a later time point since following vaccination. Microneedle immunized non-pregnant mice were fully protected, when challenged 80 days after vaccination. Intramuscularly immunized mice were 86% protected when challenged 4 weeks after vaccination but none of the animals survived when challenged 80 days after vaccination. These results suggest that microneedle immunized mice are protected for prolonged periods of time while the protective immunity in intramuscularly immunized mice decreases with time.

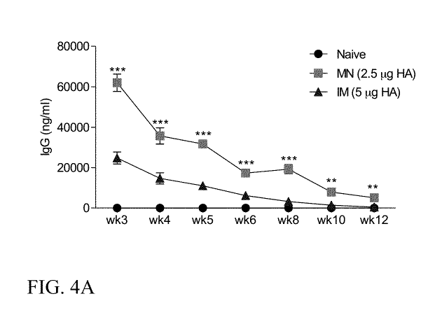

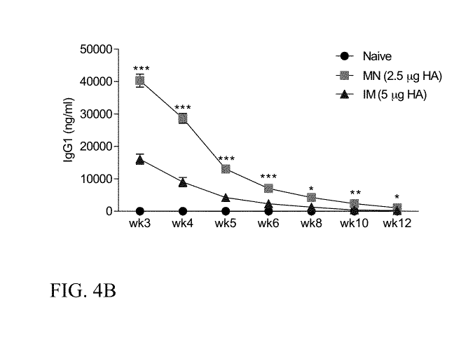

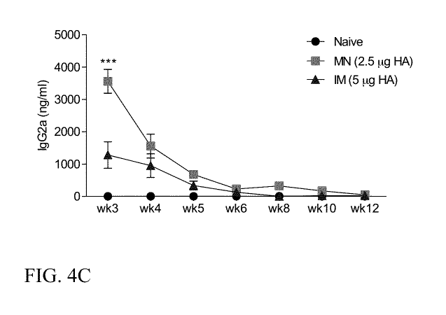

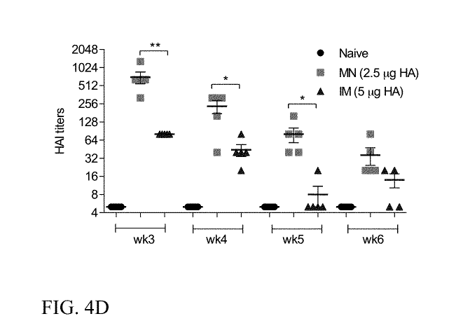

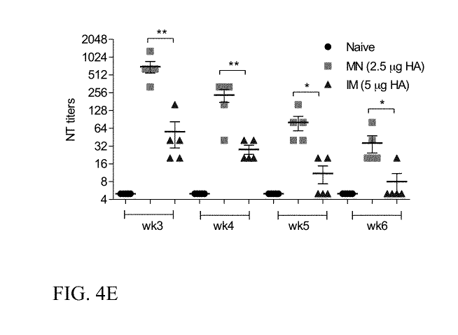

Pups born to mothers immunized during pregnancy using microneedles have higher levels of influenza specific antibody titers in the sera as compared to pups that were born to mice immunized via the intramuscular route during pregnancy. According to CDC it is not recommended to administer influenza vaccine to infants younger than 6 months of age. However, immunized mothers can transfer antibodies to their offspring through breast milk. In order to evaluate the levels of vaccine specific antibody levels in sera of pups born to immunized mice sera was collected form pups on weeks 3, 5, 6, 8, 10 and 12 after birth. The first blood collection was done on the day the pups were separated from the mothers, and the following blood collections were done to determine the duration of influenza specific antibodies in the sera. Consistently with what was observed with the immunized mothers, specific antibody titers were higher in pups born to mice that were immunized with the microneedles during pregnancy. The IgG, IgG1, and IgG2a antibody levels were significantly higher (p<0.0001) in 21-day old pups born to mothers immunized with microneedles as compared to the mice immunized via the intramuscular route (FIGS. 4A, 4B and 4C). Although specific antibody titers were decreasing with time, the titers in pups born to mothers immunized with microneedles were consistently higher as compared to titers observed in the other groups of pups. Consistent with those observations, significantly higher functional (HAI and NT) antibody titers were observed in pups born to microneedle immunized mothers (FIGS. 4D and 4E) on weeks 3, 4, 5, 6 after birth (p=<0.001-0.05)

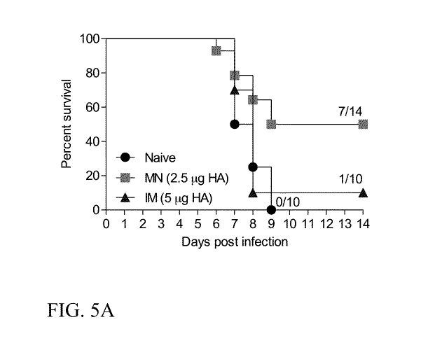

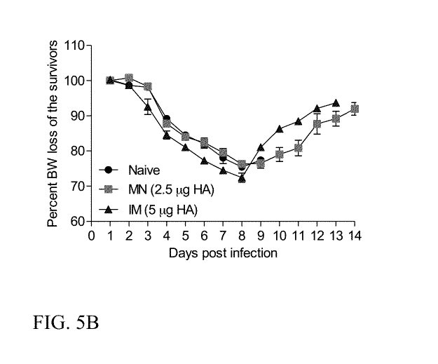

Immunization of pregnant mice using microneedles results in improved protection of their offspring. Six week-old pups were challenged with 5×LD50 of A/Brisbane/57/07 (H1N1) virus intranasally and observed for signs of morbidity and mortality for 14 days. Ten percent survival rate was observed in pups born to pregnant mice immunized with 5 μg of HA intramuscularly, while 50% survival rate was observed in pups born to mice immunized with 2.5 μg of HA using microneedles (FIGS. 5A and 5B).

All publications and patents and literature specifically mentioned herein are incorporated by reference for all purposes.

It is understood that this invention is not limited to the particular methodology, protocols, materials, and reagents described, as these may vary. It is also to be understood that the terminology used herein is for the purpose of describing particular embodiments only, and is not intended to limit the scope of the present invention, which will be encompassed by the appended claims.

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprises,” “comprising”, “containing,” “including”, and “having” can be used interchangeably.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever.

All of the features disclosed in this specification may be combined in any combination. Each feature disclosed in this specification may be replaced by an alternative feature serving the same, equivalent, or similar purpose.

What is claimed is:

1. A method for preventing, treating or reducing the effects of influenza infection in a subject, comprising administering an influenza vaccine to the skin of the subject.

2. The method of claim 1, wherein the influenza vaccine does not require cold storage or cold transport.

3. The method of claim 1, wherein the influenza vaccine is an influenza subunit vaccine.

4. The method of claim 1, wherein the influenza vaccine comprises an influenza subunit vaccine at an effective dose of from 1.5 to 5 μg of HA.

5. The method of claim 1, wherein the influenza vaccine comprises an influenza subunit vaccine at an effective dose of 2.5 μg of HA.

6. The method of claim 1, wherein the administration is intradermal or microneedle.

7. The method of claim 6, wherein the microneedle administration is a microneedle patch.

8. The method of claim 1, wherein the subject is pregnant.

9. The method of claim 8, wherein offspring born to immunized mothers have higher levels of specific anti-influenza antibodies in sera than offspring born to mothers immunized with a double dose of the same vaccine via an intramuscular route.

10. The method of claim 8, further comprising:
administering an influenza vaccine by placing a microneedle patch containing the vaccine on the skin of the subject;
holding the patch in place to allow the vaccine to dissolve into the skin.

11. The method of claim 10, wherein the microneedles of the patch penetrate the skin and the patch is held in place for at least 10 minutes.

12. The method of claim 10, wherein no vaccine reconstitution is required prior to administration, and the microneedle vaccination patch is stored at ambient temperature.

13. The method of claim 10, wherein at least a three-fold higher level of influenza-specific antibodies is induced in pregnant subjects than for intramuscular administration in pregnant subjects.

14. The method of claim 10, wherein at least a five-fold higher level of influenza-specific antibodies is induced in non-pregnant subjects than for intramuscular administration in non-pregnant subjects.

15. The method of claim 10, wherein at least a six-fold higher level of influenza-specific antibodies IgG2a is induced in a pregnant subject as compared to intramuscular administration in a non-pregnant subject.

16. The method of claim 1, wherein the duration of protection against influenza infection is greater than for intramuscular administration.

17. The method of claim 1, wherein the duration of protection against influenza infection is twice as long as for intramuscular administration.
